(19) United States
(12) Patent Application Publication
(10) Pub. No.: US 2009/0274675 A1
(43) Pub. Date: Nov. 5, 2009

(54) Migraine tonic

(76) Inventor: Govindan Gopinathan, Oradel, NJ (US)

Correspondence Address: Govindan Gopinathan, M.D., 680 Briarwood Court, Oradel, NJ, 07649, US

(21) Appl. No.: 12/214,563

(22) Filed: Jun. 21, 2008

Related U.S. Application Data
(60) Provisional application No. 61/126,245, filed on May 3, 2008.

(51) Int. Cl. A61K 31/122; A61P 25/06

(52) U.S. Cl. 424/94.1

(57) ABSTRACT

This invention is proposing a combination of a system of migraine headache remedies from non-prescription pharmaceuticals in the form of an aqueous drinkable tonic utilizing as the component foundation the main ingredient Ubiquinone (Co-enzyme-Q10) coupled with one or more of the following non-prescription natural or synthesized pharmaceuticals to include but are not inclusive of Tanacetum Parthenium, or Hypericin and/or Hyperforin, Petasin, Magnesium Citrate, and Riboflavin, the selection of which is determined upon the user and the user's other medications.

### CROSS-REFERENCES TO RELATED APPLICATIONS

This application is based on provisional application No. 61/126,245 filed May 5, 2008 and claim is made for the benefit of the filing date of the provisional application.

### STATEMENT REGARDING FEDERALLY SPONSORED RESEARCH AND DEVELOPMENT

Not Applicable.

### REFERENCE TO A “Microfiche Appendix”

Not Applicable.

### BACKGROUND OF THE INVENTION

1. Field of the Invention

Migraine is a very common disorder, more than 28 million Americans suffer from migraine, another 12 million at least, suffer from migraine, not diagnosed. It is a chemical disorder of the brain, generating an “electrical storm” which slowly spreads from one part of the brain to another. There are effective treatment for acute migraine, by using prescription medications, which patients accept without resistance. However, there is resistance on the part of the patients for using prescription drugs for long term prevention of migraine. There seems to acceptance among the general public for the use of alternate medical treatments, mostly herbal medications, in particular, for long term use.

The term Migraine originated from a term coined by the Italian anatomist Galen, “hemicrania”. This term was later dubbed into latin as hemigranea and migranea; eventually its translation into French, migraine, gained popularity and still continues to be so. What Galen described was a collection of symptoms comprising: one sided severe, recurrent, headache; photophobia (sensitivity to light),vomiting, relief by lying down in a dark environment and sleep. Unfortunately, what Galen described is not true in almost 50% of Migraine sufferers. Headache could be bilateral, very often throbbing, need not be severe. Not uncommonly patients only complain of a band like “pressure” around the head and neck, the scalp could be tender and hence many patients may just call it a “tension headache”. Many suffer from chronic nasal congestion and stuffiness, prompting the patient to say: “I don't have migraine, but I suffer from sinus headaches!” Pain not uncommonly, could be felt behind the eye, inside the ear, or inside a tooth. Very often neck stiffness, mostly on one side, is the dominant symptom, making even doctors mis-diagnose the condition as cervical disc disease or cervical arthritis. It would be more practical to consider any recurring headache as Migraine, unless other vice proved.

Most often migraine is an inherited trait, Nearly 94% of women suffering from Migraine have a family history. Every three out of four migraine sufferer is a woman.

Environmental factors (temperature, humidity) food (most often coffee, chocolate, aging cheese and red wine) are culprits to trigger migraine. Emotional stress, lack of sleep and not eating in time (hypoglycemia) are potent causes to trigger a migraine attack.

The first speculation of migraine as a brain dysfunction came from the publication of Living in 1873 “A contribution to the pathology of Nerve Storms”. This was an astonishing speculation, at a time when electro-physiology was at it's infancy. Living truly believed migraine is due to a “nerve storm” in the brain. His further speculation and analogy of migraine to epilepsy makes one surmise, what he meant by the word “nerve storm” is actually an electrical storm (an abnormal electrical discharge from nerve cells.)

Migraine is a “syndrome” meaning a cluster of symptoms, which are notoriously recurrent. Many times this starts in early childhood or in the teens, patients dismiss them as tension headache or allergy headaches or even more often as “sinus headaches”. The repeated occurrence of headaches (one sided or both sides), light sensitivity (photophobia), sound sensitivity (phonophobia) and odor sensitivity (osmophobia) nausea, nasal stuffiness, neck stiffness, scalp tenderness, etc. are pointers leading to migraine than any other type of headache. A positive family history helps confirming the diagnosis. Many women experience this headache has a tendency to occur just before or during the menstrual period, a characteristic not seen in other headaches.

Migraine is a “chemical” illness. Migraine could be broadly defined as an “inherited trait, triggered by several internal and environmental factors” The neuro-transmitter Serotonin plays a major role in the migraine process, the trigger factor, which could be emotional upheavals, exposure to food such as red wine, aging cheese, chocolate, etc. and exposure to change in environment, could trigger migraine. The trigger factor causes release in the brain of a variety of chemical substances (peptides), the most important ones being CGRP (Calcitonin Gene Related Protein) Substance P, Bradykinin etc. These are vasoactive peptides, causing dilatation and/or constrction of blood vessels. The serotonin receptors located along the inner lining of the blood vessels in the brain and in the coverings (meninges) of the brain, play an important role in the genesis of the symptoms of migraine, headache being the most prominent symptom.

Other neurotransmitters are also implicated in the migraine process. Release of nor-epinephine causes fast heart rate, sweating and a sense of anxiety.; all these are effectively blocked by beta blocker drugs. Dopamine release could cause signs of acute depression, nausea and vomiting; a drug called metachlorpramide (Reglan) blocks these effects. Other neurotransmitters like glutamate and Gaba Amino Butyric Acid (GABA) are also implicated in the pathophysiology of migraine.

2. Description of the Related Art

There are also many patents that have been assigned for migraine headache solutions. Such past efforts where compounds utilize herbal or other off-shelf non regulated components can be catalogued into three basic categories: the feverfew group, the Taurine/CoQ10 group, and the analgesic group. Among those within the feverfew group include U.S. Pat. No. 4,758,433 which demands the addition of a lactone to an extract of the feverfew plant, while others such as U.S. Pat. No. 6,038,999 and No. 6,500,450 require the addition of a magnesium salt and riboflavin. The appropriate magnesium salts are varied in some instances and are not dose specific. U.S. Pat. No. 6,312,736 talks about the use of a feverfew and ginger extract along with root and bark analgesics while U.S. Pat. No. 6,967,033 utilizes alternative herb components added to a feverfew extract and a ginger extract and U.S. Pat. No. 7,192,614 simply uses the two component feverfew and ginger extracts. Wherein these do use broad herbal components, their specifications and claims do not require the sub-components and related dosages specific to defined medical practice.

The taurine and CoQ10 enzyme group is demonstrated with U.S. Pat. No. 4,962,121 wherein taurine, a type of component that produces an anxiolytic-like effect and is used for the treatment of cats, is the main ingredient while U.S. Pat. No. 6,403,116 uses the enzyme CoQ10 with methyl sulfonyl methane and citric acid and leaves out the taurine. U.S. Pat. No. 6,465,517 requires both taurine and CoQ10 as the main component coupled with creatine and carnitine. Percentages of doses are given in some cases, but the formulations do not represent that which is defined within the invention of this patent.

The last group represent those patents which define compounds which require the use of analgesics. This is a practice to be avoided when there are unknown and undesirable reactions that may occur among the users of various forms of analgesic components. These merely reflect already available pain reducing compounds which are already available. U.S. Pat. No. 5,538,959 combines an analgesic agent with a magnesium salt and an effervescent component while U.S. Pat. No. 6,642,243 is simply a combination of an already famous mixture and including pseudoephedrine, acetaminophen, and caffeine. U.S. Pat. No. 6,770,263 is much more broad in that it teaches a solution containing aspirin, ibuprofen, and naproxen with an herbal analgesic plus several herbal extracts including feverfew and butterbur. No specific dosages are provided and no specific sub-components. Such use of a broad spectrum of components is dangerous in that toxic elements can be included within the gathered herbal groups. From the broadest teaching to the simplest we find U.S. Pat. No. 7,018,983 where a double dose of aspirin is combined with a double dose of tea (which contains caffeine components), and honey and apple cider vinegar. A common folk remedy which now holds a patent.

We see from the teachings found within many U.S. patents that highly specific component dosages and the use of sub-components are entirely or partially disregarded and that the formulations do not provide a variety of formulations suited to the needs of the public at large.

### BRIEF SUMMARY OF THE INVENTION

A study of the existing and prior art seems to reveal the fact that no attempt has been made so far to combine a group of naturally available drugs utilizing as the basic component known as the Co-enzyme-Q-10, which has distinct and pharmacologically well recognized roles to play in the treatment of migraine. Other formulations are known to use the Co-enzyme-Q-10 as an added ingredient, but not as a foundation and chemically structural basis for the total formula. Since the demographic statistics indicate more than half of the migraine sufferers do not seek formal medical help and use OTC drugs to treat their migraine by themselves, this patent application outlines a combination of one to five components added to the Co-enzyme-Q-10, all naturally available, and OTC (Over The Counter), in varying combinations. This would help all segments of the migraine population to use this natural combination remedy. One must bear in mind that the integrity of the final product is determined upon the purity of the components and the availability of the components. This invention is therefore not limited to natural extracts but allows the use of laboratory synthesized components that are accepted as exact substitutes and chemically equal to the natural component.

The dosages mentioned in this patent application are not fixed regimens, in practical use, could be used in higher or lower dosages of each drug. Also, these six components would be used in combination of all six, or less than six: for example: two, three, four, in combination with the basic Co-enzyme-Q-10. The ingredients of this “Migraine Herbal Tonic” would be therefore:

Co-enzyme-Q10 plus a formulation of various components including:

A. Extracts from Feverfew

B. An extract of St. John's Wort

C. Extracts from Butterbur Root

D. The Magnesium Citrate salt

E. Riboflavin known as vitamin B2

### DETAILED DESCRIPTION OF THE INVENTION

This patent relates to the use of the Co-enzyme-Q-10, which has distinct and pharmacologically well recognized roles to play in the treatment of migraine and a combination of six other medications, all naturally available, and OTC (Over The Counter), in varying combinations.

The dosages mentioned in this patent application are not fixed regimens, in practical use, could be used in higher or lower dosages of each drug. Also, these six drugs would be used in combination of all six drugs, or less than six drugs, for example: two, three, four, or five drugs in combination. The ingredients of this “Migraine Herbal Tonic” would be:

The Co-enzyme-C-10 plus a formulation of various components including:

A. Extracts from Feverfew

B. An extract of St. John's Wort

C. Extracts from Butterbur Root

D. Magnesium Citrate as the salt of preference

E. Riboflavin known as vitamin B2.

The Co-enzyme Q10 (CoQ10) known as Ubiquinone, is essentially a vitamin or vitamin-like substance. Vitamins are defined as organic compounds essential in minute amounts for normal body function, acting as co-enzymes or precursors to co-enzymes. The biosynthesis of CoQ10 from the aminoacid tyrosine, is a complex process involving at least eight vitamins and several trace elements. Co Q10 is the coenzyme for at least three mitochondrial enzymes (complexes 1, II and III). Mitochondrial enzymes of the oxidative phosphorylation pathway are vital for the production of high energy phosphate and adenosine triphosphate (ATP) upon which all cellular function depend. The electron and proton transfer functions of the quinine ring are of fundamental importance to all forms of life. In its reduced form CoQ10 is a potent antioxidant.

Co Q10 was first isolated from beef heart mitochondria by Dr. Frederick Crane of Wisconsin, U.S.A. in 1957. The same year, Professor Morton from U.K. isolated a compound from vitamin A deficient rat liver, it was identified the same compound as Co Q10. In 1958, Professor Karl Folkers at Merck, Inc. defined the chemical structure of Co Q10 as: 2,3 dimethoxy-5 methyl-6 decaprenyl benzoquinone, and was the first to synthesize it by fermentation process. In 1960 Professor Yamamura of Japan first used CoQ 7, a related compound to Co Q10, in clinical practice to treat patients with congestive heart failure. In 1966, Mellors and Tappel demonstrated that reduced form of CoQ10 is an effective anti-oxidant. In 1972 Gian Paolo Littarru of Italy along with Karl Folkers of Merck, documented CoQ10 deficiency in human heart disease. In 1978 Peter Mitchell received the Nobel Price for his contribution to the understanding of biological energy transfer through the formulation of the chemiosmotic theory, which includes the vital protonmotive role of CoQ10 in the energy transfer systems.

In 1986 Karl Folkers received the Priestly Medal from the American Chemical Society, and in 1990 President Bush senior, awarded him the National Medal of Science, for his work on CoQ10 and other vitamins.

Since the Japanese were able to produce CoQ10 in commercial quantities, its value in clinical medicine has been documented by clinical trials in Heart failure, Cancer, AIDS, Parkinson's Disease, prevention of Atherosclerosis and in immunological diseases. Scientists believe, the clinical applications of CoQ10 is only just emerging and it holds the potential to alter our current thinking of therapeutics.

The Role of CoQ10 in treatment of Migraine is just being documented (Neurology February 2004). Its role as a free radical scavenger, antioxidant, mediator of energy transfer and stabilizing cell membranes and mitochondria, all may be at play in its role as a treatment for migraine. CoQ10 was initially used in doses of 30 to 45 mg per day in the trials for congestive heart failure.

In this invention I am proposing Ubiquinone (Co-enzyme-Q-10) as the foundation and main ingredient, in combination with other components. The Co-enzyme-Q-10 Uniquinone has the major role to play in the proposed migraine preventive herbal combo: “Migraine Tonic” and is to be administered in the amount of 200-400 mg. per unit dose of the formula combination.

The extract of Feverfew to be used is from an extracted group called Parthenolides. The botanical name of Feverfew is Chrysanthemum parthenium and its medical extract is a Parthenolide. The group of Parthenolides are sesquiterpene lactones which are found in Feverfew and the particular Parthenolide to be used is known as Tanacetum parthenium. The feverfew plant is a perennial herb with an erect, branched, downy and leafy stem and many alternate, yellowish, pinnate to bi-pinnate leaves. The fruit is a ribbed achene without pappus; all components of this plant is strongly aromatic. This plant most likely is native of south-east Europe, Asia Minor and the Caucasus. It is available in most of Europe including the British Isles and also the Mediterranean region. The common name Feverfew, is a corruption of the Latin word “febrifugia”, an implication of the plants use formerly, as a febrifuge: fever reducing medication.

An infusion of the dried herb was used as a stomachic, sedative, disinfectant, and anti-spasmodic. Very recently Feverfew has been given attention as a migraine drug, especially in Britain, where it is being studied in clinical settings. It's migraine relieving effect could be attributed to its action as an antagonist to prostaglandins, an inflammation causing peptide. Protaglandins are implicated in the perpetuation of aseptic inflammation of the meninges (coverings of the brain) and other tissues around the head and neck, causing migraine headache to continue for days. The amount of natural or laboratory produced Tanacetum parthenium to be used is 1.0 mg or less per dosage unit. However, since some findings indicate that the extract from feverfew should not be taken in pregnancy, an alternate formulation found within this invention should be utilized for women in pregnancy.

The extracts of St. John's Wort that are known to treat migraine are Hypericin and Hyperforin. One of the aftermath and/or co-morbidity of migraine is depression. The chemical substrate for this is the low serotonin levels in the brain, which results from repeated episodes of migraine. Excretion of serotonin metabolites in the urine are significantly elevated during a migraine attack, indicating rapid turnover of serotonin reserves in the brain. In patients with an inherent propensity for depression, (endogenous depression) the serotonin depletion could precipitate and perpetuate depression, unless the levels are corrected by treatment. The majority of patients with migraine related depression have mild or moderate depression. Cases with severe depression, must be treated seriously by an experienced psychiatrist.

St. John's Wort is a perennial herb with a stout creeping rhizome which bears clumps of erect branched stems. The flowers are yellow, and the sepals and petals are black dotted, especially at the edges. The fruit is capsule shaped. It is commonly seen throughout Europe, including Britain. Its name starting as “St. John's” came from the fact that the plant was used by the Knights of St. John of Jerusalem to heal the wounds of Crusaders. The flowering stem is used in medicine. Their components include tannins, and flavonoid glycosides like hypericin, hyperflorin, rutin, and catechol and resins. It has been used to treat chronic inflammation, and menstrual disorders. This plant has been used for medicinal purposes for some 2000 years.

Recently there has emerged great interest in St. John's wort, as an anti-depressant. It has been a popular anti-depressant for more than 15 years in Europe, in Germany alone physicians write 3 million prescriptions a year; this is 25 times more than the number of prescriptions they write for Prozac.

St. John's Wort is getting attention in the U.S as well: Rob McCaleb of the Herb Research Foundation called it “the premier herb for treating moderate depression”. In 1994, the prestigious Journal of Geriatric Psychiatry and Neurology, devoted an entire issue containing 17 research articles on St. John's Wort. One study tracked the herb's effect on 3,250 patients with mild to moderate depression 80% either felt better or became completely symptom free after four weeks of treatment. In an article published in British Medical Journal in August of 1996, 23 controlled studies involving 1757 depressed patients were reviewed: the conclusion was St. John's wort worked nearly 3 times better than placebo.

How safe is St. John's wort? In a study of 3,250 depressed patients, only 2.4% experienced side effects, which included restlessness; gastrointestinal irritations, and mild allergic reactions. Purdue University herb expert Varro Tyler noted the common prescription antidepressants like Prozac, causes more serious side effects than this herb. However, there is concern high doses of this herb could cause photosensitivity, like in sheep who ingested large quantities of St. John's Wort. No one has reported photosensitivity in patients using this herb for depression. The dosages indicated for this invention are 10 mg. or less per unit dose or Hypericin and/or 750 mg. or less per unit dose of Hyperforin either naturally extracted or laboratory synthesized.

The extracts of St. John's Wort St. John's wort may cause increased sensitivity to sunlight. Other side effects can include anxiety, dry mouth, dizziness, gastrointestinal symptoms, fatigue, headache, or sexual dysfunction.

Research shows that St. John's Wort interacts with some drugs. The herb affects the way the body processes or breaks down many drugs; in some cases, it may speed or slow a drug's breakdown. Drugs that can be affected include:

Antidepressants

Birth control pills

Cyclosporine, which prevents the body from rejecting transplanted organs

Digoxin, which strengthens heart muscle contractions

Indinavir and possibly other drugs used to control HIV infection

Irinotecan and possibly other drugs used to treat cancer

Warfarin and related anticoagulants

When combined with certain antidepressants, St. John's wort may increase side effects such as nausea, anxiety, headache, and confusion.

For the above specific cautions indicated, it is therefore advised that an alternate formulation within this invention be utilized that does not include the extracts of St. John's Wort.

The extracts of Butterbur root are Petasin and lospetasin. A perennial herb with a creeping horizontal rhizome, seen to sprout in early spring. Flowers are reddish violet and the fruit is an achene with a pappus of long hairs. This herb grows throughout in Europe, including British Isles. The name Butterbur came from the fact its large leaves were once used to wrap up butter in hot weather. The roots are mainly used in treatment of migraine. In the Homeopathic Pharmacopia, the tincture of the fresh plant is used to treat “neuralgia”.

A clinical study of the root of Butterbur was done by Richard Lipton, from Albert Einstein College of Medicine, Bronx, N.Y., and published in the popular journal NEUROLOGY on December 2004. 254 migraine patients with two to six attacks per month were studied with a dosage regimen of 75 mg twice daily over a period of 4 months. 48% reduction of headache frequency was noted, which was statistically significant, and more effective than placebo. The drug was well tolerated.

It must be remembered that the extracts for use in this invention come from the root of Butterbur only and not from any other component of the plant. The natural extract of Petasin and/or Isopetasin or the laboratory synthesis of these extracts are to be used in dosages of 7 mg. or less of each per unit dose.

The Magnesium salt known as Magnesium Citrate. Magnesium is an element (Mg) with an atomic number 12 and atomic weight of 24.3050. It oxidizes to magnesia, a bio-element and it's many salts have clinical applications.

Magnesium plays an important role in membrane physiology, in maintaining membrane electrical polarity and calcium ion transport across cell membranes. Its deficiency causes seizures in humans. It is essential for cardiac conduction of electrical impulses, skeletal muscle relaxation and neuro-chemical transmission. In the brain, decrease in mg levels causes neuronal irritability and excitation, leading to frank “electrical storm” or seizure activity.

One of the current views on migraine is, it is a clinical phenomenon of neuronal excitation, the emerging electrical excitation spreads as a “wave” from one pole of the brain to the other. Migraine patients experience often, loss of speech, numbness of one side of face, then arm, body and lower limb. The cause of this “march” of migraine symptoms is attributed to the “spreading wave of excitation” generated by one group of neurons. It is noteworthy Living speculated in 1873, migraine is a “nerve storm”.

Use of magnesium is useful to control this neuronal excitability, which is one factor that perpetuates migraine. I have used it as an intravenous injection in intra-partum (in labor) and post-partum (after delivery) women suffering from severe migraine, with dramatic effect. In this invention the unit dosage of Magnesium Citrate to be used is 250 to 500 mg. per total formulation dosage.

The vitamin B2 known as Riboflavin. Riboflavin is a water soluble, heat stable, factor of the Vitamin B—Complex. Components of this vitamin are co-enzymes of the flavodehydrogenases, which plays important role in oxidative metabolic pathways, generating energy bonds. Daily requirement for adult men is 1.7 mg and for adult women 1.3 mg. Higher doses are required during pregnancy and lactation. Dietary sources include, green vegetables, liver, kidney, wheat germ, diary products, and fish.

A deficit of mitochondrial energy metabolism is considered top play a role in the genesis of migraine. Riboflavin plays a vital role in mitochondrial oxidative metabolism. Riboflavin is also considered to have an anti-inflammatory effect, by inhibiting the inflammatory peptide, prostaglandin. These hypotheses was tested with several clinical trials, both in the U.S and in Europe. Patient' improvement in migraine headache were statistically significant. Prediction is riboflavin would take a key role in the control of migraine. Dosages to be used in this invention are from 2 to 8 mg. of Riboflavin (vitamin B2) per unit dose of formulation.

I claim:

1. a method and composition for reducing the risk of migraine headaches in a human subject in need thereof, comprising the administration of an effective amount of an aqueous composition having all components dispersed or dissolved therein comprising Ubiquinone (Co enzyme-Q10) in the amount of 200-400 mg, and Tanacetum Parthenium of 1.0 mg or less.

2. A method for reducing the risk of migraine headaches of claim 1 wherein said said Tanacetum Parthenium is a sub-component of Parthenolide which is an extract of a natural plant such as, but not inclusive of, feverfew.

3. A method for reducing the risk of migraine headaches of claim 1 wherein said Tanacetum Parthenium is synthetically produced in a pharmaceutical laboratory.

4. A method for reducing the risk of migraine headaches of claim 1 wherein said aqueous composition is in the form of a contained drinkable solution with a unit dosage of 5.0 to 10.0 fluid ounces.

5. A method for reducing the risk of migraine headaches of claim 1 further comprising the inclusion of a soluble form of a Magnesium salt.

6. A method for reducing the risk of migraine headaches of claim 5 further comprising Magnesium Citrate in the amount of 250-500 mg. per unit dose as the said Magnesium salt.

7. A method for reducing the risk of migraine headaches of claim 1 further comprising the inclusion of Petasin in the amount of 7 mg. per unit dose.

8. A method of reducing the risk of migraine headaches of claim 7 wherein said Petasin is an extract of a natural plant such as but not inclusive of the root of Butterbur.

9. A method of reducing the risk of migraine headaches of claim 7 wherein said Petasin is synthetically produced in a pharmaceutical laboratory.

10. A method for reducing the risk of migraine headaches of claim 7 further comprising the inclusion of Isopetasin in the amount of 7 mg. per unit dose.

11. A method of reducing the risk of migraine headaches of claim 10 wherein said Isopetasin is an extract of a natural plant such as but not inclusive of the root of butterbur.

12. A method of reducing the risk of migraine headaches of claim 10 wherein said Isopetasin is synthetically produced in a pharmaceutical laboratory.

13. A method for reducing the risk of migraine headaches of claim 1 further comprising the inclusion of Hypericin in the amount of 10 mg. per unit dose.

14. A method of reducing the risk of migraine headaches of claim 13 wherein said Hypericin is an extract of a natural plant such as but not inclusive of St. John's Wort.

15. A method of reducing the risk of migraine headaches of claim 13 wherein said Hypericin is synthetically produced in a pharmaceutical laboratory.

16. A method of reducing the risk of migraine headaches of claim 13 further comprising the inclusion of Hyperforin in the amount of 750 mg. per unit dose.

17. A method of reducing the risk of migraine headaches of claim 16 wherein said Hyperforin is an extract of a natural plant such as but not inclusive of St. John's Wort.

18. A method of reducing the risk of migraine headaches of claim 16 wherein said Hyperforin is synthetically produced in a pharmaceutical laboratory.

19. A method for reducing the risk of migraine headaches of claim 1 further comprising the inclusion of Riboflavin which is also known as vitamin B2 in the amount of 2-8 mg. per unit dose.

20. A method for reducing the risk of migraine headaches of claim 5 further comprising the inclusion of Petasin in the amount of 7 mg. per unit dose.

21. A method of reducing the risk of migraine headaches of claim 20 wherein said Petasin is an extract of a natural plant such as but not inclusive of the root of Butterbur.

22. A method of reducing the risk of migraine headaches of claim 20 wherein said Petasin is synthetically produced in a pharmaceutical laboratory.

23. A method for reducing the risk of migraine headaches of claim 20 further comprising the inclusion of Isopetasin in the amount of 7 mg. per unit dose.

24. A method of reducing the risk of migraine headaches of claim 23 wherein said Isopetasin is an extract of a natural plant such as but not inclusive of the root of Butterbur.

25. A method of reducing the risk of migraine headaches of claim 23 wherein said Isopetasin is synthetically produced in a pharmaceutical laboratory.

26. A method for reducing the risk of migraine headaches of claim 5 further comprising the inclusion of Hypericin in the amount of 10 mg. per unit dose.

27. A method of reducing the risk of migraine headaches of claim 26 wherein said Hypericin is an extract of a natural plant such as but not inclusive of St. John's Wort.

28. A method of reducing the risk of migraine headaches of claim 26 wherein said Hypericin is synthetically produced in a pharmaceutical laboratory.

29. A method of reducing the risk of migraine headaches of claim 26 further comprising the inclusion of Hyperforin in the amount of 750 mg. per unit dose.

30. A method of reducing the risk of migraine headaches of claim 29 wherein said Hyperforin is an extract of a natural plant such as but not inclusive of St. John's Wort.

31. A method of reducing the risk of migraine headaches of claim 29 wherein said Hyperforin is synthetically produced in a pharmaceutical laboratory.

32. A method for reducing the risk of migraine headaches of claim 5 further comprising the inclusion of Riboflavin which is also known as vitamin B2 in the amount of 2-8 mg. per unit dose.

33. A method for reducing the risk of migraine headaches of claim 23 further comprising the inclusion of Riboflavin which is also known as vitamin B2 in the amount of 2-8 mg. per unit dose.

34. A method for reducing the risk of migraine headaches of claim 29 further comprising the inclusion of Riboflavin which is also known as vitamin B2 in the amount of 2-8 mg. per unit dose.

35. A method for reducing the risk of migraine headaches of claim 23 further comprising the inclusion of Hypericin in the amount of 10 mg. per unit dose.

36. A method of reducing the risk of migraine headaches of claim 35 wherein said Hypericin is an extract of a natural plant such as but not inclusive of St. John's Wort.

37. A method of reducing the risk of migraine headaches of claim 35 wherein said Hypericin is synthetically produced in a pharmaceutical laboratory.

38. A method of reducing the risk of migraine headaches of claim 35 further comprising the inclusion of Hyperforin in the amount of 750 mg. per unit dose.

39. A method of reducing the risk of migraine headaches of claim 38 wherein said Hyperforin is an extract of a natural plant such as but not inclusive of St. John's Wort.

40. A method of reducing the risk of migraine headaches of claim 38 wherein said Hyperforin is synthetically produced in a pharmaceutical laboratory.

41. A method for reducing the risk of migraine headaches of claim 38 further comprising the inclusion of Riboflavin which is also known as vitamin B2 in the amount of 2-8 mg. per unit dose.

42. A method for reducing the risk of migraine headaches of claim 10 further comprising the inclusion of Riboflavin which is also known as vitamin B2 in the amount of 2-8 mg. per unit dose.

43. A method for reducing the risk of migraine headaches of claim 10 further comprising the inclusion of Hypericin in the amount of 10 mg. per unit dose.

44. A method of reducing the risk of migraine headaches of claim 43 wherein said Hypericin is an extract of a natural plant such as but not inclusive of St. John's Wort.

45. A method of reducing the risk of migraine headaches of claim 43 wherein said Hypericin is synthetically produced in a pharmaceutical laboratory.

46. A method of reducing the risk of migraine headaches of claim 43 further comprising the inclusion of Hyperforin in the amount of 750 mg. per unit dose.

47. A method of reducing the risk of migraine headaches of claim 46 wherein said Hyperforin is an extract of a natural plant such as but not inclusive of St. John's Wort.

48. A method of reducing the risk of migraine headaches of claim 46 wherein said Hyperforin is synthetically produced in a pharmaceutical laboratory.

49. A method for reducing the risk of migraine headaches of claim 16 further comprising the inclusion of Riboflavin which is also known as vitamin B2 in the amount of 2-8 mg. per unit dose.

50. A method for reducing the risk of migraine headaches of claim 46 further comprising the inclusion of Riboflavin which is also known as vitamin B2 in the amount of 2-8 mg. per unit dose.

51. A method for reducing the risk of migraine headaches of claim 5 wherein said aqueous composition is in the form of a contained drinkable solution with a unit dosage of 5.0 to 10.0 fluid ounces.

52. A method for reducing the risk of migraine headaches of claim 10 wherein said aqueous composition is in the form of a contained drinkable solution with a unit dosage of 5.0 to 10.0 fluid ounces.

53. A method for reducing the risk of migraine headaches of claim 16 wherein said aqueous composition is in the form of a contained drinkable solution with a unit dosage of 5.0 to 10.0 fluid ounces.

54. A method for reducing the risk of migraine headaches of claim 19 wherein said aqueous composition is in the form of a contained drinkable solution with a unit dosage of 5.0 to 10.0 fluid ounces.

55. A method for reducing the risk of migraine headaches of claim 23 wherein said aqueous composition is in the form of a contained drinkable solution with a unit dosage of 5.0 to 10.0 fluid ounces.

56. A method for reducing the risk of migraine headaches of claim 29 wherein said aqueous composition is in the form of a contained drinkable solution with a unit dosage of 5.0 to 10.0 fluid ounces.

57. A method for reducing the risk of migraine headaches of claim 32 wherein said aqueous composition is in the form of a contained drinkable solution with a unit dosage of 5.0 to 10.0 fluid ounces.

58. A method for reducing the risk of migraine headaches of claim 33 wherein said aqueous composition is in the form of a contained drinkable solution with a unit dosage of 5.0 to 10.0 fluid ounces.

59. A method for reducing the risk of migraine headaches of claim 34 wherein said aqueous composition is in the form of a contained drinkable solution with a unit dosage of 5.0 to 10.0 fluid ounces.

60. A method for reducing the risk of migraine headaches of claim 38 wherein said aqueous composition is in the form of a contained drinkable solution with a unit dosage of 5.0 to 10.0 fluid ounces.

61. A method for reducing the risk of migraine headaches of claim 41 wherein said aqueous composition is in the form of a contained drinkable solution with a unit dosage of 5.0 to 10.0 fluid ounces.

62. A method for reducing the risk of migraine headaches of claim 42 wherein said aqueous composition is in the form of a contained drinkable solution with a unit dosage of 5.0 to 10.0 fluid ounces.

63. A method for reducing the risk of migraine headaches of claim 46 wherein said aqueous composition is in the form of a contained drinkable solution with a unit dosage of 5.0 to 10.0 fluid ounces.

64. A method for reducing the risk of migraine headaches of claim 49 wherein said aqueous composition is in the form of a contained drinkable solution with a unit dosage of 5.0 to 10.0 fluid ounces.

65. A method for reducing the risk of migraine headaches of claim 50 wherein said aqueous composition is in the form of a contained drinkable solution with a unit dosage of 5.0 to 10.0 fluid ounces.

66. A method and composition for reducing the risk of migraine headaches in a human subject in need thereof, comprising the administration of an effective amount of an aqueous composition having all components dispersed or dissolved therein comprising Ubiquinone (Co enzyme-Q10) in the amount of 200-400 mg, and Magnesium Citrate with a dosage of 250-500 mg administered twice in a 24-hour period.

67. A method and composition for reducing the risk of migraine headaches in a human subject in need thereof, comprising the administration of an effective amount of an aqueous composition having all components dispersed or dissolved therein comprising Ubiquinone (Co enzyme-Q10) in the amount of 200-400 mg, and Riboflavin (Vitamin B2) with a dosage of 2-8 mg administered twice in a 24-hour period.
